(19) United States
(12) Patent Application Publication
(10) Pub. No.: US 2016/0074275 A1
(43) Pub. Date: Mar. 17, 2016

(54) Constant hot temperature flat massage ball

(71) Applicant: Kun Yuan Tong, Suwanee, GA (US)

(72) Inventor: Kun Yuan Tong, Suwanee, GA (US)

(21) Appl. No.: 14/121,489

(22) Filed: Sep. 11, 2014

(51) Int. Cl. A61H 15/02; A61F 7/08; A61F 7/00

(52) U.S. Cl. CPC A61H 15/02; A61F 7/007; A61F 7/08; A61F 2007/0054

(57) ABSTRACT

The constant hot temperature flat massage ball comprises one top shell-shaped receptacle, one bottom shell-shaped receptacle and a central heating unit which is either a hot water bag, which has circulating water tube, a water pump and a precision temperature control hot water reservoir equipped with a thermostat, or an electric heating pad, which has an electric cord and a thermostat. The top shell-shaped receptacle has a central recess to store the heating unit and a circular female threaded wall at the upper portion of central recess. The bottom shell-shaped receptacle also has a central recess to store the heating unit, a circular male threaded embankment above the central recess to screw into the above-mentioned circular female threaded wall and a groove which is built transversely across its wall and the above-mentioned male threaded embankment to admit the above mentioned circulating water tube and electric cord. The water bag of the above-mentioned central heating unit is heated by hot water, which is pre-heated to 100 to 104 degree Fahrenheit controlled by a thermostat in precision temperature hot water reservoir, through the circulating water tube pumped by a water pump. The central electric heating pad has a thermostat which is preferably adjusted at 100 to 104 degree Fahrenheit. The constant hot temperature flat massage ball is constantly heated by hat water bag or heating pad. Therefore, it can be used for continuously massage with massage body cream or lotion without interruption.

### THE BACKGROUND OF THE INVENTION

The hot massage stone will cool off in about two minutes and needs to be reheated by hot water again. The presently invented constant hot temperature flat massage ball which shapes like a massage stone and is constantly heated either by a hot water bag which is stored inside the flat massage ball and constantly heated by circulating hot water, which circulates from a precision temperature water reservoir with a thermostat adjusted at 100 to 104 degree Fahrenheit, or by an electric heating pad which is stored inside the flat metal ball and controlled by a thermostat to adjust its temperature at 100 to 104 degree Fahrenheit. Therefore, the constant hot temperature flat massage ball can maintain a hot temperature, preferably at 100 to 104 degree Fahrenheit, for continuous message with body cream. Massager has to test temperature of flat massage ball.

### THE BRIEF SUMMARY OF THE DEVICE

Hot massage stones will cool off in about two minutes and need to be reheated by hot water again. The presently invented constant hot temperature flat massage ball is kept at a constantly hot temperature either by a hot water bag or an electric heating pad at a fixed hot temperature, about 100 to 104 degree Fahrenheit. The conventional massage stones have to be removed after cooling off in two minutes and replaced by other heated massage stones.

### THE BRIEF DESCRIPTION OF THE DRAWINGS

FIG. 1 is a perspective view of a constant hot temperature flat massage ball which is heated by a hot water bag.

FIG. 2 is a perspective view of components of constant hot temperature flat massage ball which is heated by a hot water bag.

FIG. 3 is a perspective view of constant hot temperature flat massage ball which is heated by an electric heating pad.

FIG. 4 is a perspective view of components of constant hot temperature flat massage ball which is heated by an electric heating pad.

### THE DETAILED DESCRIPTION OF THE INVENTION

References are as follow:
1. Self-heating massage stone by Dominic Orlando. U.S. Pat. No. 8,715,210. Mr. Orlando disclosed a heating electric element which is heated by rechargeable battery with no thermostat for temperature control. The massage stone may be overheated or under heated.
2. Massage stone by Sylvie Hennessy. U.S. Pat. No. D 560,810 S. Mr. Hennessy disclosed an ornamental design for a massage stone.
3. Pocket body warmer by Toya et al. U.S. Pat. No. 8,278,606.
4. Personal vibratory massager by James D. TerMeer. U.S. Pat. No. D 620,601 S.
5. The ornamental design for a massager by Jarry. U.S. Pat. No. D 626,656 S.
6. Hollow massage device containing heating or cooling agent by John Olov Linden. U.S. Pat. No. 1,380,986.
7. Device providing a heated place for animals by Stammelman. U.S. Pat. No. 5,261,352.
8. Electric frying pan having automatically controlled heating means by Ivar Jepson. U.S. Pat. No. 2,744,995.

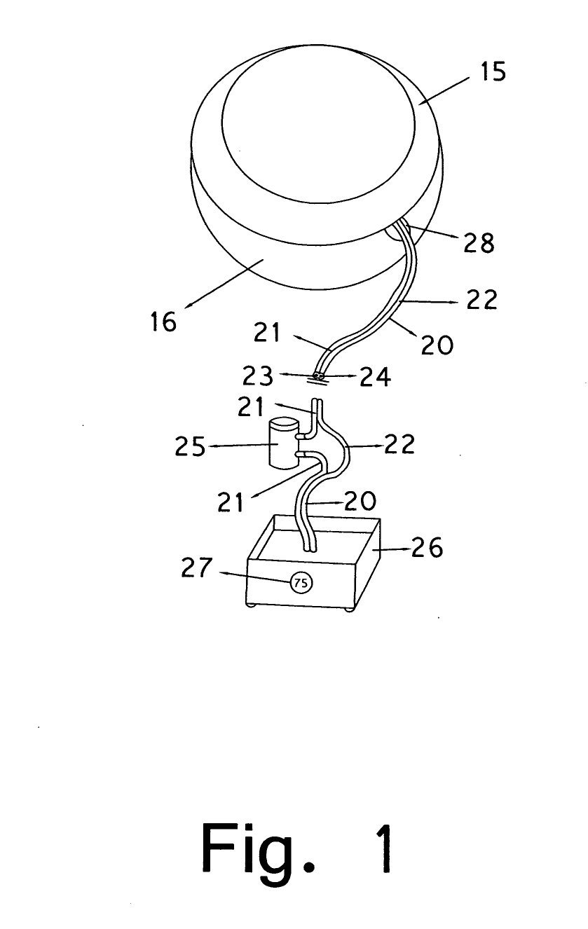

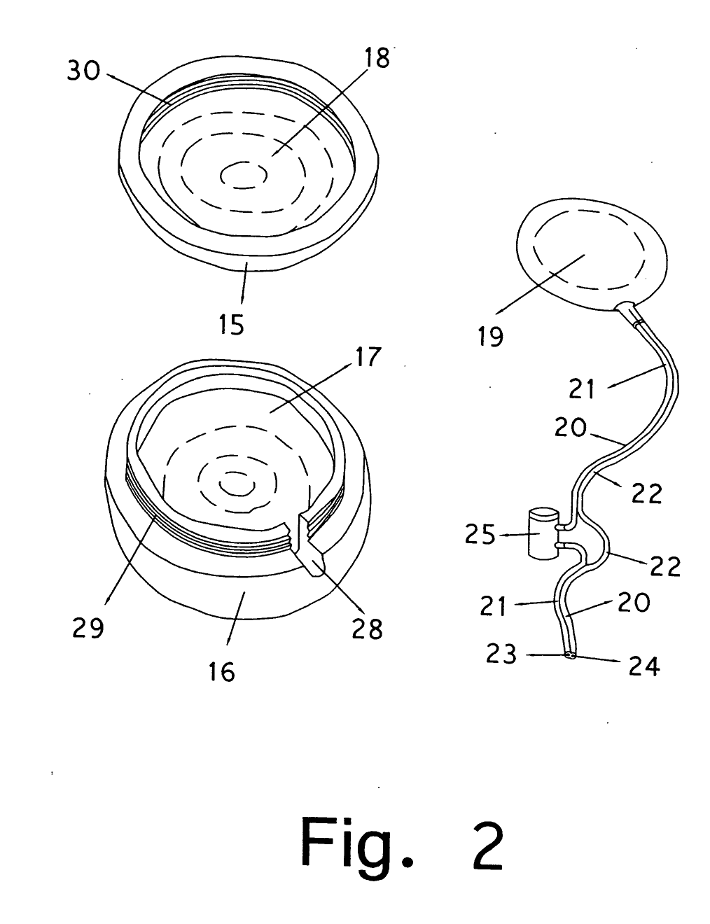

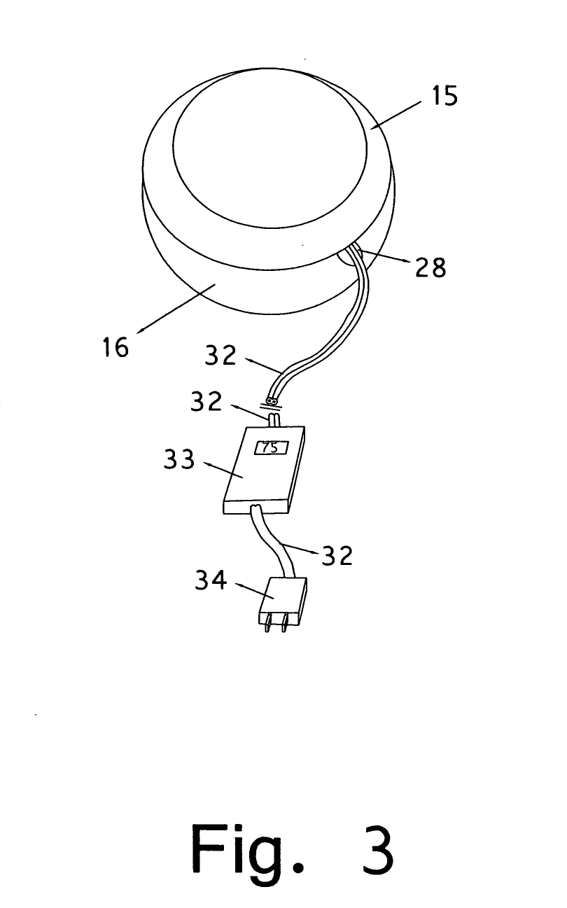

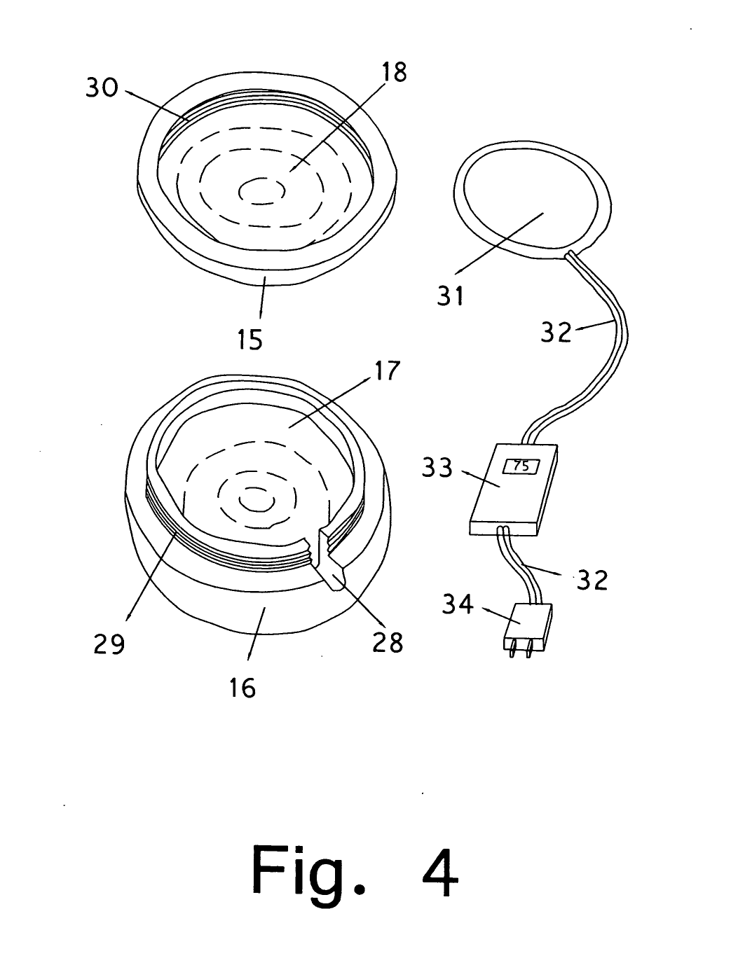

Referring now in detail to the drawing numeral 11 of FIG. 1 is a constant hot temperature flat massage ball which shapes like a massage stone and is heated by a hot water bag (abbreviated as flat massage ball-HWB hereafter). Numeral 12 of FIG. 2 are components of flat massage ball-HWB 11. Number 13 of FIG. 3 is a constant hot temperature flat massage ball which shapes like a massage stone and is heated by an electric heating pad (abbreviate as flat massage ball-EHP hereafter). Numeral 14 of FIG. 4 are components of flat massage ball-EHEP 13. Numeral 15 of FIGS. 1, 2, 3 and 4 is top shell-shaped receptacle of flat massager ball-HWB 11 and flat massage ball-EHP 13. Numeral 16 of FIGS. 1, 2, 3 and 4 is the bottom shell-shaped receptacle of massage flat ball-WHB 11 and massage flat ball-EHP 13. Top shell-shaped receptacle 15 and bottom shell-shaped receptacle 16 can be made of gold or silver or copper or steel or aluminum or ceramic materials. Numeral 17 of FIGS. 2 and 4 is central recess of bottom shell-shaped receptacle 16. Numeral 18 is central recess of top shell-shaped receptacle 15. Numeral 19 of FIG. 2 is hot water bag. Hot water bag 19 is stored inside central recess 18 and central recess 17. Numeral 20 of FIGS. 1 and 2 is water tube. Numeral 21 of FIGS. 1 and 2 is inward water tube of water tube 20. Numeral 22 of FIGS. 1 and 2 is outward water tube of water 20. Water tube 20 contains inward water tube 21 and outward water tube 22. Numeral 23 of FIG. 2 is canal of inward water tube 21. Numeral 24 of FIG. 2 is canal of outward water tube 22. Numeral 25 of FIGS. 1 and 2 is one-way water pump. Numeral 26 of FIG. 1 is precision temperature water reservoir. Numeral 27 of FIG. 1 is thermostat which is preferably adjusted at 100 to 104 degree Fahrenheit. Numeral 28 of FIGS. 1, 2, 3 and 4 is groove of bottom shell-shaped receptacle 16. Groove 28 can admit water tube 20. Numeral 29 of FIGS. 2 and 4 is male threaded circular embankment of bottom shell-shaped receptacle 16. Male threaded circular embankment 29 is built around the central recess 17 as illustrated in FIGS. 2 and 4. Numeral 30 of FIGS. 2 and 4 is female threaded circular wall. Female threaded circular wall 30 is built at upper portion of central recess 18. Male threaded circular embankment 29 can screw snugly and tightly into female threaded circular wall 30. When water pump 25 is turned on it pumps hot water from precision temperature water reservoir 26 trough inward water tube 21 into hot water bag 19 and then the cooler water returns from water bag 19 through outward water tube 22 into precision temperature hot water reservoir 26 to be reheated. Hot water bag 19 transmits heat to top shell-shaped receptacle 15 and bottom shell-shaped receptacle 16 to keep them at a constant hot temperature. The thermostat 25 is preferably adjusted at 100 to 104 degree Fahrenheit. If hotter water temperature or lower temperature is desired the thermostat 27 of precision temperature water reservoir 26 can be adjusted to a desired higher temperature or lower temperature. Number 31 of FIG. 4 is electric heating pad. Number 32 of FIGS. 3 and 4 is electric cord. Number 33 of FIGS. 3 and 4 is thermostat. Number 34 of FIGS. 3 and 4 is electric plug. Electric heating pad 31 is stored inside central recess 17 and central recess 18. Electric cord 32 can pass through groove 28. After electric plug 34 is plugged in wall socket and the thermostat 33 is set at 100 degree Fahrenheit the electric heating pad 31 starts to heat to reach a temperature of 100 degree Fahrenheit and transmits heat to top shell-shaped receptacle 15 and bottom shell-shaped receptacle 16 to keep them at a constant hot temperature at around 100 degree Fahrenheit.

1. The constant hot temperature flat massage ball comprising a flat massage ball and a heating unit.

2. The said flat massage ball of claim 1 comprising one top shell-shaped receptacle which has a top central recess and a female circular threaded wall at the upper portion of said top central recess and one bottom shell-shaped receptacle which has a bottom central recess, a male circular threaded embankment, which is built above the said bottom central recess, and a groove, which is built transversely across its wall and the said male circular threaded embankment, wherein the said male circular threaded embankment screws snugly and tightly into said female circular threaded wall.

3. The said heating unit of claim 1 comprising a water bag, one connecting double-canal water tube, a one-way water pump and a precision temperature hot water reservoir equipped with a thermostat. Wherein the said water bag is stored inside said top central recess and bottom central recess of claim 2. Wherein the said connecting double-canal water tube connects to the said water bag at one end and one free end to be merged into the said precision temperature hot water reservoir. Wherein the said water pump is installed in the middle portion of the said double-canal water tube. Wherein the said precision temperature hot water reservoir is equipped with a thermostat.

4. The said heating unit of claim 1 comprising an electric heating pad, one electric cord, one thermostat and one plug. Wherein the said electric heating pad is stored inside the said top central recess and bottom central recess of claims 2 and 3. Wherein the said electric cord connects the said electric heating pad at one end and connects the said plug at the other end. Wherein the said thermostat is installed at the middle portion of said electric cord.
